(19) United States
(12) Patent Application Publication
(10) Pub. No.: US 2024/0344249 A1
(43) Pub. Date: Oct. 17, 2024

(54) KNITTED STRAIN SENSOR

(71) Applicant: TECHNISCHE UNIVERSITEIT DELFT, Delft (NL)

(72) Inventors: Kaspar Maria Bonaventura Jansen, Den Haag (NL); Beyza Bozali, Den Haag (NL); Joris Johannes Franciscus Van Dam, Berkel en Rodenrijs (NL); Linda Plaude, Amsterdam (NL)

(21) Appl. No.: 18/681,717

(22) PCT Filed: Aug. 5, 2022
(86) PCT No.: PCT/NL2022/050459

§ 371 Date: Feb. 6, 2024

(30) Foreign Application Priority Data
Aug. 6, 2021 (NL) 2028936

(51) Int. Cl. D04B 1/10; D04B 1/16; D04B 1/18; D04B 1/24; G01L 1/22

(52) U.S. Cl. CPC D04B 1/102; D04B 1/16; D04B 1/18; D04B 1/24; G01L 1/2287; D10B 2401/061; D10B 2401/16; D10B 2401/18; D10B 2403/02431

(57) ABSTRACT

Embodiments in this disclosure relate to a knitted strain sensor element comprising an electrically conducting yarn and an elastic yarn. The elastic yarn has a Young's modulus that is substantially lower than the electrically conducting yarn's Young's modulus. The knitted strain sensor element is knitted using a knit stitch pattern comprising knitted stitches and purled stitches on each course. preferably a rib stitch pattern, more preferably a 1×1 rib stitch pattern. The electrically conducting yarn and the elastic yarn are knitted together using a plated knitting technique forming a knitted fabric. the electrically conducting yam forming a core of the knitted fabric and the elastic yarn forming surface of the knitted fabric. Sensors. textiles and garments comprising such knitted strain sensor elements are also disclosed.

### CROSS-REFERENCE TO RELATED APPLICATION(S)

The present application is a Section 371 National Stage Application of International Application No. PCT/NL2022/050459, filed Aug. 5, 2022 and published as WO 2023/014224 A1 on Feb. 2, 2023, and further claims priority to Netherlands patent application no. 2028936, filed Aug. 6, 2021.

### FIELD OF THE INVENTION

The disclosure relates to knitted strain sensor elements, and to strain sensors, textiles, and garments comprising such sensor elements.

### BACKGROUND

Significant attention has been devoted to so-called ‘smart’ textiles in recent years. Smart textiles may be used for various applications such as textile-based antennas, energy harvesting, electromagnetic shielding, and health monitoring. Integrating various smart functions in garments helps to capitalize upon the intrinsic qualities of textiles such as comfort, stretchability, and washability. Thus, textiles provide appropriate platforms as a host for human interaction because they fit the shape of the human body, allowing for easy implementation to the functionality of the electrical components incorporated within. A precondition for many such applications is an accurate and reliable strain sensor that can be integrated in textiles, e.g., for use in garments. Strain sensors commonly used for mechanical engineering applications are typically limited to strain not larger than 1%. For on-body applications, however, a strain sensor must be capable of measuring strains up to 30-40%. In addition, for unobtrusive monitoring of, e.g., physiological parameters, the sensors need to be breathable, washable, and stretchable.

Knitted strain sensors may achieve seamless integration into garments. Thus, they are promising candidates for applications such respiratory monitoring or elbow and knee motion monitoring, because of their generally good elastic recovery and stretchability. However, knitted structures often demonstrate unstable characteristics, which typically result in high hysteresis values, poor sensing performance, and a narrow working range. Consequently, they are currently not commonly integrated into garments, but are mostly applied in a patch form.

To be able to obtain accurate measurements with knitted strain sensors, they must have a low hysteresis. Preferably, they also show a linear resistance vs. strain relationship over a working range of up to 40% strain, high sensitivity (i.e., a high gauge factor), and sensor properties that are stable over time. Therefore, considerable effort has gone into creating strain sensors that incorporate conductive yarns within a non-conductive fabric construction (e.g., knitted or stitched strain sensors), and that have such desirable properties.

However, known sensors typically suffer from hysteresis, leading to a low accuracy of the resulting signal. Furthermore, sensors made of impregnated textiles usually have a low breathability and may show severe degradation over time due to, e.g., repeated washing of the sensors.

Hence, there is a need in the art for a strain sensor with low hysteresis and a linear response over a working range of at least 40%.

### SUMMARY

It is an objective of the embodiments in this disclosure to reduce or eliminate at least one of the drawbacks known in the prior art.

In a first aspect, the invention relates to a knitted strain sensor element comprising an electrically conducting yarn and an elastic yarn. The elastic yarn has a Young's modulus that is substantially lower than the electrically conducting yarn's Young's modulus. The knitted strain sensor element is knitted using a knit stitch pattern comprising knitted stitches and purled stitches on each course, e.g., a rib stitch pattern such as a 1×1 rib stitch pattern. The electrically conducting yarn and the elastic yarn are knitted together using a plated knitting technique forming a knitted fabric, the electrically conducting yarn forming a core of the knitted fabric and the elastic yarn forming surfaces of the knitted fabric.

It has been found that a strain sensor based on such a knitted strain sensor element is substantially hysteresis-free, having a normalized electrical hysteresis of less than 0.05, wherein the normalized electrical hysteresis is defined as the maximum strain difference at equal electrical resistance between loading and unloading curves, normalised by the strain range applied during the hysteresis measurement. The knitted strain sensor has a large working range of at least about 40%, and a highly linear response over essentially the whole working range. Moreover, the knitted strain sensor was found to be washable and durable.

The plated structure where the conductive yarn is knitted together with an elastic yarn ensures an elastic textile with a low mechanical hysteresis. A knit stitch pattern with knitted and purled stitches on each course, in particular a rib stitch pattern, more in particular a 1×1 rib stitch pattern, ensures a high working range and, through its high elastic recovery, helps reducing the mechanical and electrical hysteresis of the sensor. It is noted that the stretch of the sensor is primarily caused by deformation of the knitting pattern, rather than by the elasticity of the used conductive yarns. Indeed, there is no need for the conductive yarns to be (very) elastic.

When a knit stitch pattern comprising knitted stitches and purled stiches on each course is used, and in particular when a knit stich pattern comprising knitted wales and purled wales is used, the resulting fabric has a yarn that is predominantly on the inside of the fabric (forming its core) and a yarn that is predominantly on the outside of the fabric (forming its surfaces). Thus, the core of the fabric may also be referred to as the inside of the fabric. and the surface of the fabric may also be referred to as the outside of the fabric. In general, the yarn that is on top during the knitting of a course, i.e., furthest away from the already knitted fabric, is the yarn that is (predominantly) on the inside of the knitted fabric and the bottom yarn is the yarn that is (predominantly) on the outside of the knitted fabric. It has been found that, surprisingly, a plated knit stitch pattern with the conductive yarn on the inside (core) has a much lower hysteresis than the same plated knit stitch pattern with the conductive yarn on the outside (surface). This is probably due to the number of contact points within a conductive course and, especially, between conductive courses.

It is generally thought that the overall resistance of the knitted sensor is affected by the contact pressure and contact area between loop base and loop heads of adjacent stitches of the conductive yarn as well as overlapping loop heads. The first effect (base to head contact) is not stitch-specific and occurs for many knit stitches. The second effect, however, is specific for the knitting structure defined in claim 1 with the conductive yarn forming the core of the fabric (i.e., at the inside). During stretching, these loops slide over each other, resulting in observable and reproducible resistance changes. The co-knitted elastic yarns at the outsides (surfaces) provide the necessary contact pressure.

In this disclosure, the term ‘conductive yarn’ refers to an electrically conducting yarn, unless specified otherwise.

As used herein, ‘hysteresis’ refers to normalized electrical hysteresis, unless otherwise specified. Hysteresis in strain sensors is measured on a tensile tester in combination with a device which records the resistance. The strain is typically applied by extending the strain sensor. Unless otherwise specified, the strain is applied in a course direction of the knitted fabric. The normalized electrical hysteresis is defined as the maximum strain difference at equal electrical resistance between loading and unloading curves, normalised by the strain range applied during the hysteresis measurement. The measurement should cover at least the full working range. The normalized electrical hysteresis may be determined, e.g., based on a plot of the measured resistance versus applied strain.

As used herein, ‘knitted strain sensor’ refers to a strain sensor comprising a knitted sensor element. The knitted strain sensor may also comprise non-knitted elements, e.g., a microprocessor and connective wiring.

In an embodiment, the knitted strain sensor element comprises one or more adjacent courses of the electrically conducting yarn. For example, the knitted strain sensor element may comprise at most twenty, at most ten, at most six, or between two to four inclusive, adjacent courses of the electrically conducting yarn. Sensors with high numbers of adjacent conductive courses, e.g., more than twenty, may have a smaller working range than sensors with fewer adjacent conductive courses. Therefore, in an embodiment, the sensor has most ten adjacent conductive courses. A single course sensor has relatively few contact points and may show a lower linearity than a sensor with multiple adjacent courses of the conductive yarn.

In an embodiment, the knitted strain sensor element comprises one or more, e.g., 5 or more, or 10 or more adjacent courses of the elastic yarn on either side of the one or more courses of the electrically conducting yarn. The courses of elastic yarn on either side of the one or more courses of the electrically conducting yarn may increase the elastic recovery of the fabric, reducing the mechanical and electrical hysteresis.

In an embodiment, the electrically conducting yarn has an electric resistance in the range of 5 to 10000 Ω/m inclusive, e.g., in the range 10-1000 Ω/m inclusive. A resistance that is higher may lead to a gauge factor that is too low, while a resistance that is lower may lead to a very noisy signal.

In an embodiment, the electrically conducting yarn comprises a non-conductive core with an electrically conductive coating. For example, the coating may comprise silver, and the non-conductive core may comprise a synthetic polyamide or a polyester, e.g., nylon. Coated yarns have a good washability and are typically more stable over time than many other types of conductive yarns, such as, e.g., yarns based on stainless steel filaments. In particular, silver-coated yarns have a good electric conductivity. A non-conductive core comprising a synthetic polyamide such as nylon results in a strong and resilient yarn.

In an embodiment, the elastic yarn has an elastic recovery of more than 95%, e.g., than 99% or even substantially 100%. As used herein, elastic recovery, or resilience, is a measure of the ability of an elastomer to return to its original shape when a previously applied mechanical load is removed. The elastic yarn may comprise a synthetic polyamide and/or a synthetic polyether-polyurea copolymer such as elastane. The elastic yarn may comprise between 10-25% inclusive fibers of elastane or another synthetic polyether-polyurea copolymer, e.g., between 15-20% inclusive. The elastane fibers may comprise more than 70% of soft segment blocks. Elastane is also known as spandex. The elastic yam may have an elastic modulus of 0.1-0.5 cN/tex as determined using the ASTM-D2731-15 method. Use of a yarn with a high elastic recovery, such as yarns comprising elastane, may result in a knitted structure with a low mechanical hysteresis, which in turn may lead to or at least facilitate a low electrical hysteresis.

In a further aspect, the invention relates to a textile, e.g., a knitted textile, comprising a knitted strain sensor element as described above. The knitted strain sensor element may be embedded in the textile. Such a textile can be connected to or integrated in, e.g., a mechanical construction such as a robot arm.

In a further aspect, the invention relates to a strain sensor comprising a knitted strain sensor element as described above, a measuring unit for measuring the electrical resistance of the knitted strain sensor element, and a processing unit coupled to the measuring unit, the processing unit being configured to determine a strain or a property derived from the strain, based on the measured electrical resistance. This way, output may be obtained from the sensor element that can be used to, e.g., determine the amount of stretch of the sensor element. The measuring unit may comprise a voltage divider and a voltage source.

In a further aspect, the invention relates to a garment comprising a knitted strain sensor element or a strain sensor as described above, wherein the knitted strain sensor element is adapted for sensing a physiological signal, e.g., one of: a respiration signal, limb compression, skin deformation, stretching, or body motion. The garment can be a close-fitting garment.

Additionally or alternatively, the knitted strain sensor may be adapted for determining a position of a body part, e.g., a limb, of a user. This way, the sensor may provide input for a feedback system for a user performing exercises, e.g., in a physiotherapy context. This way. the user can, e.g., track progress or check whether the exercises are being performed correctly. Additionally or alternatively, the sensor may provide input for a feedback system for monitoring body posture. This way, the feedback system can, e.g., warn the user of a bad posture. In this embodiment, the garment may comprise a plurality of sensor elements configured to work together.

Such a garment could also be use in a Virtual Reality context to determine, e.g., a pose of a user in order to properly render the pose in the virtual environment.

The invention will be further illustrated with reference to the attached drawings, which schematically will show embodiments according to the invention. It will be understood that the invention is not in any way restricted to these specific embodiments. Identical reference signs refer to identical, or at least similar elements.

### BRIEF DESCRIPTION OF THE DRAWINGS

Aspects of the invention will be explained in greater detail by reference to exemplary embodiments shown in the drawings, in which:

FIG. 1A depicts an electrical resistance versus strain relationship for a knitted strain sensor with a high electric hysteresis and FIG. 1B depict an electrical resistance versus strain relationship for a knitted strain sensor with a low electric hysteresis according to an embodiment of the invention;

FIG. 2A schematically depicts two knit stitches;

FIG. 3A and 3B schematically depict a non-plated 1×1 rib stitch pattern;

FIG. 4A-4C schematically depict, respectively, a top view, a front view, and a perspective view of a plated 1×1 rib stitch pattern according to an embodiment of the invention.

FIG. 5A-5C schematically depicts a yarn feeder, FIG. 5B schematically depicts a needle in a needle bed and FIG. 5C schematically depicts a conductive yarn and an elastic yarn, all as used for plated knitting according to an embodiment of the invention;

FIG. 6 schematically depicts a strain sensor according to an embodiment of the invention;

FIG. 7A-7C schematically depict sensor elements according to embodiments of the invention; and

FIG. 8 schematically depicts a garment comprising a knitted strain sensor element according to an embodiment of the invention.

### DETAILED DESCRIPTION

The embodiments in this disclosure describe a knitted strain sensor with a low electrical hysteresis.

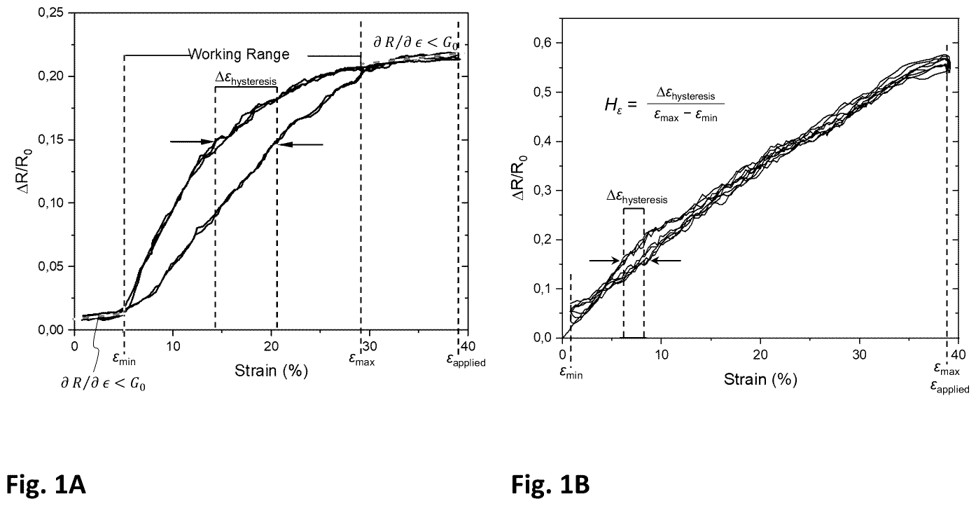

FIG. 1A depicts an electrical resistance versus strain relationship for a knitted strain sensor with a high electric hysteresis not according to an embodiment of the invention. In particular, FIG. 1A depicts a graph representing an electrical resistance of a knitted strain sensor as function of the strain during a tensile extension relaxation test. The knitted strain sensor is extended to 140% of its original size and subsequently relaxed to allow the sensor to return to its original shape. This cycle is repeated multiple times. It can be seen that the electrical resistance of the sensor is different during extension than during relaxation. The maximum of this difference is the hysteresis. The (normalized) hysteresis may be determined per cycle and subsequently averaged over a series of cycles. If the electrical resistance is used to determine the strain, this leads to an uncertainty in the determined strain. For example, in this case, a relative resistance change of 15% may indicate an extension (and hence strain) of either 14% or 20.5%.

In general, the size of the hysteresis effect increases with the applied strain range of the sensor. In a sensor, the full applied strain range cannot always be used for sensing. The strain range available for sensing is referred to as the working range of the sensor, and can be defined as the region where the change in resistance due to a change in strain, i.e., the gauge factor, exceeds a predetermined amount, that is, the region for which ∂R/∂ϵ>G₀. If the derivative of the resistance with respect to the strain is too small, the uncertainty in the strain becomes too large. In practice, the effective working range of a sensor may depend on the required accuracy.

As used in this disclosure, electrical hysteresis is defined as the maximum difference in strain corresponding to the same resistance (or relative resistance change), divided by the applied strain range during the measurement. Typically, the applied strain range covers at least the intended working range of the sensor element. Generally, the applied strain does not exceed the maximum working range. Thus, the electrical hysteresis H_{ϵ} may be determined by:

$${H}_{ϵ} = \frac{{Δϵ}_{hysteresis}}{{ϵ}_{applied}} \qquad ( 1 )$$

Therefore, the depicted example has a hysteresis of approximately H_{ϵ}=0.065/0.39=17%. This definition follows, e.g., K. M. B. Jansen, ‘Performance evaluation of knitted and stitched textile strain sensors’, Sensors 20 (2020) 7236, which is hereby incorporated by reference. Unless otherwise specified, as used herein, ‘hysteresis’ refers to electrical hysteresis.

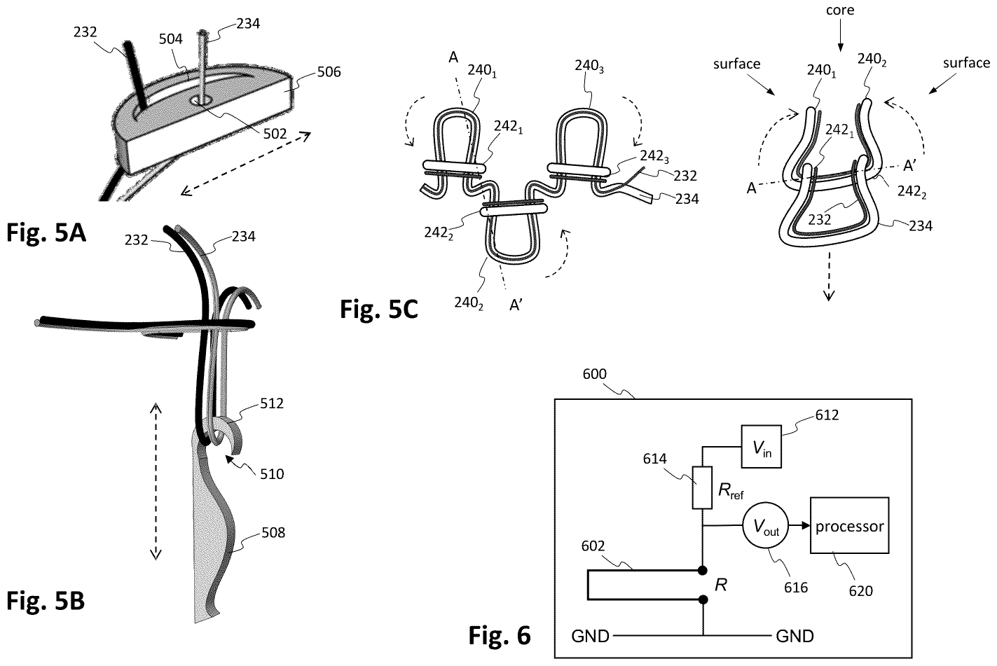

The sensor whose resistance-strain curve is depicted in FIG. 1A is a knitted strain sensor comprising an electrically conducting yarn and an elastic yarn. The electrically conducting yarn is Shieldex® 235/36 dtex Z 130 HC+B, with a resistivity of 600 Ω/m. The elastic yarn is a Yeoman elastomeric-white-1 yarn which consists of 81% nylon and 19% elastane with a dtex value of 192. The sensor is knitted using a 1×1 rib stitch pattern with a 19×10/cm² (NP9) stitch density. The electrically conducting yarn and the elastic yarn are knitted together using a plated knitting technique. The electrically conducting yarn is (predominantly) on the outside of the knitted fabric (forming its core) and the elastic yarn is (predominantly) on the inside of the knitted fabric (forming its surfaces). This will be discussed in more detail below with reference to FIG. 5A-C. The sensor is weft-knitted on a Stoll CMS 530 flat knitting machine with an E8 machine gauge and a 0.3 m/s carriage speed. The sensor is a linear sensor with two adjacent courses of the electrically conducting yarn.

Many different sensors are known in the art, with varying choices for, e.g., yarn or yarns, knit stitch pattern, knitting technique, number of adjacent conductive courses, but they all have a high hysteresis and/or a limited working range. As such, the sensor response depicted in FIG. 1A is exemplary for many prior art sensors.

FIG. 1B depicts an electrical resistance versus strain relationship for a knitted strain sensor with a low electric hysteresis according to an embodiment of the invention. The sensor is identical to the sensor described above with reference to FIG. 1A, except that the electrically conducting yarn is (predominantly) on the inside of the knitted fabric and the elastic yarn is (predominantly) on the outside of the knitted fabric. This results in the conducting yarn being mostly on the inside of the knitted fabric. The sensor has a low hysteresis, a large working range, and a high linearity. Some parameters regarding the electromechanical properties of the sensors depicted in FIGS. 1A and 1B are included in Table 1 below.

TABLE 1
Electromechanical properties of a sensor according to an
embodiment of the invention and of a reference sensor.

| Depicted | Conductive yarn | | | | |
|---|---|---|---|---|---|
| in | position | Gauge factor | Working range | Hysteresis H_{ε} | Linearity (R²) |
| FIG. 1A | outside (surface) | 0.70 ± 0.002 | 5-29% | 0.14 ± 0.018 | 0.88 ± 0.01 |
| FIG. 1B | inside (core) | 1.19 ± 0.04 | 1-39% | 0.04 ± 0.017 | 0.98 ± 0.00 |

The gauge factor G represents the sensitivity of the sensor and is defined as the (average) slope of the relative resistance change versus the applied strain, and may be defined by:

$$G = 〈 \frac{\partial R}{\partial ϵ} 〉 \approx \frac{( {R}_{max} - {R}_{0} ) / {R}_{0}}{{ϵ}_{max}} \qquad ( 2 )$$

where (⋅) denotes an average. A higher gauge factor is associated with a higher sensitivity, and thus a (for most purposes) better sensor. A high linearity makes sensor output easy to process accurately. Thus, surprisingly, a sensor as described in this disclosure may have a lower hysteresis, a higher gauge factor, a larger working range and a higher linearity than a sensor that is identical except for the positions of the conductive and elastic yarns in the fabric, corresponding to a change of position of the yarns in the yarn feeder during the knitting.

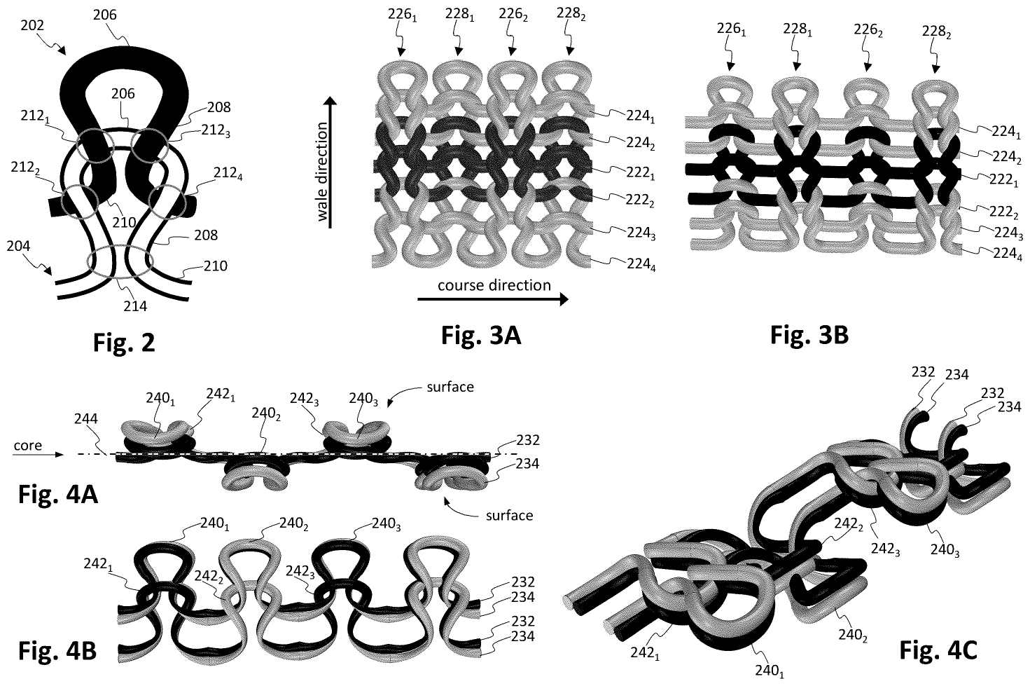

FIG. 2A schematically depicts two knit stitches. In particular, FIG. 2A schematically depicts two loops 202, 204 of adjacent courses of yarn. Each loop comprises a head 206, feet 210, and legs 208 joining the head and feet of a loop. The head of the lower loop 204 can be in contact with the feet of the upper loop 202, resulting in a plurality of inter-yarn contact points 212₁₋₄ between the two adjacent loops. The feet of a loop may also be in contact which each other, resulting in an intra-yarn contact point 214. In general, when the fabric is stretched, the intra-yarn contact points are pulled apart and have an increased resistance, whereas the inter-yarn contact points are pulled together and have a reduced resistance.

FIG. 3A and 3B schematically depict a non-plated 1×1 rib stitch pattern. FIG. 3A depicts a simple (non-plated) 1×1 rib stitch pattern in an unstretched form while FIG. 3B depicts the same pattern (though with fewer yarns) in a stretched form. The part depicted in FIG. 3A comprises two adjacent courses of a conductive yarn 222_{1,2} and four courses of a non-conductive elastic yarn 224₁₋₄, the part depicted in FIG. 2C only comprises the non-conductive elastic yarns. The depicted parts comprise two knitted wales 226_{1,2} and two purled wales 228_{1,2}. An n×m rib refers to a pattern with, alternatingly, n knitted wales and m purled wales. Thus, the depicted parts depict a 1×1 rib. As shown in FIG. 3A, the direction parallel to the course may be called the course direction, while the direction orthogonal to the courses, in this case parallel to the ribs, may be called the wale direction.

When the fabric is stretched in the course direction, the change in length is due to a change in the knit pattern, mostly due to deformation of the loop legs. Non-elastic yarns may stretch in the same way. For the conductive courses, the straightening of the loop legs may lead to an increase in electrical resistance.

FIG. 4A-C schematically depict, respectively. a top view, a front view, and a perspective view of a plated 1×1 rib stitch pattern. The depicted parts are shown in an unstretched form. For clarity, the structures are shown looser than a typical knitted strain sensor element. The depicted part comprises a conductive yarn 232 and a non-conductive elastic yarn 234. Other embodiments may use different knit stitch patterns, e.g., a 2×2 rib. The conductive yarn forms the core of the fabric while the elastic yarn forms the surfaces of the fabric, i.e., the loop heads of the conductive yarn are generally closer to a center line 244 of the course, or center plane of the fabric, than the loop heads of the elastic yarn. In other words, the conductive yarn is positioned on the ‘inside’ of the fabric while the elastic yarn is positioned on the ‘outside’ of the fabric.

FIG. 5A schematically depicts a yarn feeder of a device for plated knitting FIG. 5B schematically depicts a needle in a needle bed of a device for plated knitting, and FIG. 5C schematically depicts a conductive yarn and an elastic yarn for use in plated knitting according to an embodiment of the invention. FIG. 5A schematically depicts a yarn feeder 502 comprising a first opening 504 and a second opening 506. The device further comprises a plurality of needles 508 (only one being shown), each needle having a hook or an opening 510 and a needle head 512. The plurality of needles are positioned on one or more parallel (possibly curved) line segments, the needles on a single line segment defining a knitting bed. These one or more line segments define the course direction of the knitted fabric as explained above with reference to FIG. 3A.

When in use, the yarn feeder moves forth and back parallel to the line segments. A first yarn 232, here the conducting yarn, is fed through the first opening of the yarn feeder and a second yarn 234, here the elastic yarn, is fed through the second opening of the yarn feeder. Each of the plurality of needles moves in a direction substantially orthogonal to the line segments and engages with both the first and second yarns. When the yarn feeder has finished a course, the knitted fabric is moved in the wale direction away from the yarn feeder. In the unfinished course, the first yarn, i.e., in the depicted embodiment, the yarn that is fed through the slit opening, is placed on top, i.e., furthest from the finished fabric. The second yarn, i.e., in the depicted embodiment, the yarn that is fed through the hole opening, is placed on the bottom, i.e., closest to the finished fabric.

Other knitting machines may use a possibly different needle with a needle head and a possibly different yarn feeder with two guide openings. In such embodiments, the first yarn, i.e., the conductive yarn, is fed through the guide opening closest to the needle head and the second yarn, i.e., the elastic yarn, is fed through the opening furthest from the needle head.

When the yarn feeder is making a course, the loops are lying essentially flat on the knitting bed or beds, i.e., the loop heads extend outward from the knitted fabric. During this time, the first yarn, being fed through the opening closest to the needle heads, is positioned on top of the second yarn, being fed through the opening furthest from the needle heads. This configuration is shown on the left-hand side in FIG. 5C. When the fabric is pulled in the wale direction away from the yarn feeder, the loops move up and the first yarn is positioned on the inside of the fabric, forming its core, and the second yarn is positioned on the outside of the fabric, forming its surfaces, as shown on the right-hand side in FIG. 5C.

FIG. 5C schematically depicts a conductive yarn and an elastic yarn in a plated-knitted configuration according to an embodiment of the invention. The depicted part comprises a conductive yarn 232 and a non-conductive elastic yarn 234. During knitting. horizontal loops may be created, with the conductive yarn being positioned essentially on top of the elastic yarn. The knitted fabric is subsequently pulled down relative to the sideward pointing loops, causing the loops to fold up. Thus, the loop heads 240₁₋₃ of a first course correspond to the loop heads 242₁₋₃ of a second course adjacent to the first course. As can be seen, this results in a knitted sensor wherein the conducting yarn is mostly on the inside of the knitted fabric, forming its core, while the non-conducting elastic yarn is mostly on the outside of the knitted fabric, forming its surfaces. Other knitting machines may use slightly different plated-knitting methods, leading to the same or a very similar knitted structure with the conductive yarn on the inside, i.e., generally closest to a center line of the knitted strain sensor.

The part of the fabric comprising the sensor is knitted using a plated knitting technique combing a conductive yam and an elastic yarn. Other parts of the fabric, e.g., neighboring courses, can be knitted either using a non-plated knitting technique, using only a single elastic yarn, or using a plated knitting technique, using the elastic yam both as the first yarn and the second yarn.

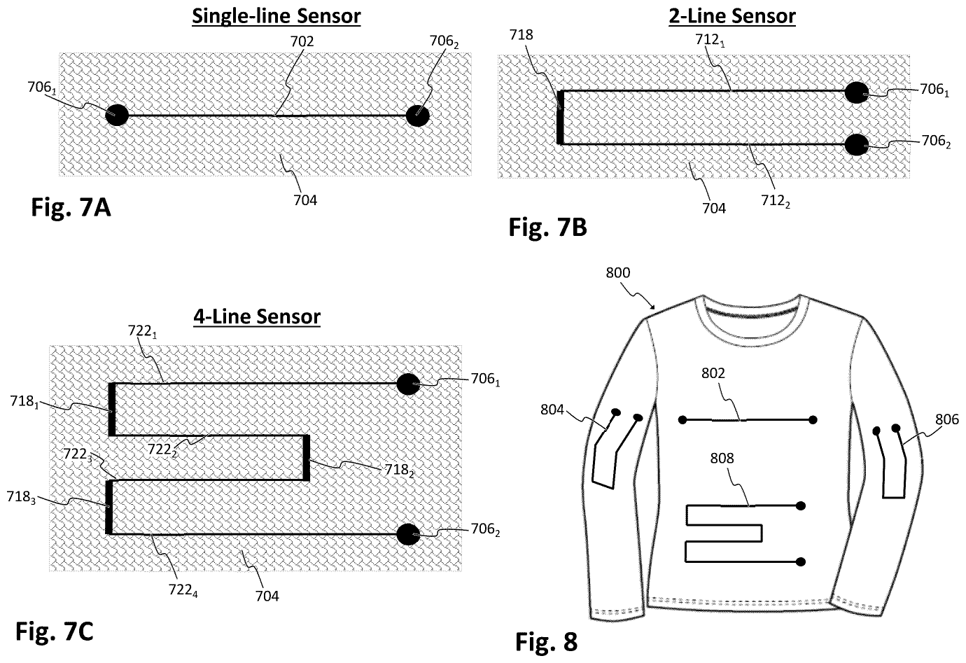

FIG. 6 schematically depicts a knitted strain sensor according to an embodiment of the invention. The knitted strain sensor 600 comprises one or more adjacent courses of a conductive yarn 602. The one or more adjacent courses of conductive yam may be referred to as a sensor line. A sensor can comprise one or more interconnected sensor lines, separated by one or more, e.g., 8 or more, or 12 or more courses of non-conductive yarn. This will be discussed in more detail below with reference to FIG. 7A-C. The conductive yarn is knitted together with an elastic yarn using a plated knitting technique. The measurement device may also comprise one or more courses of the elastic yarn (either simple or plated) on either side of the sensor line. The sensor may be configured such that a first end of the sensor is close to the second end of the sensor, e.g., by configuring the sensor in an essentially closed configuration, e.g. around a body part of a user, or by using an even number of essentially parallel sensor lines.

The measurement device further comprises a measurement unit 604 for measuring the electrical resistance of the knitted strain sensor element. The measurement unit is typically electrically connected to at least one end of the sensor element. The measurement unit may be connected using. e.g., a knitted, stitched, or externally attached conducting wire. Typically, the connecting wire has a low resistance. Generally, the connecting wire has a resistance that shows no or only negligible change in resistance in response to a strain being applied.

In a typical embodiment, the measurement unit comprises a voltage divider, but other ways to determine the resistance of the sensor can equally be used. The voltage divider comprises a voltage source 612 with a known or measured input voltage Vᵢₙ, a reference resistance 614 R_{ref} with a known resistance connected in series with the sensor element 602, and a connection 616 for determining the output voltage Vₒᵤₜ over the reference resistance. The resistance of the sensor element R may then be determined by computing:

$$R = \frac{{V}_{out}}{{V}_{in} - {V}_{out}} {R}_{ref} \qquad ( 3 )$$

The measurement device further comprises a processing unit 620 coupled to the measurement unit. The processing unit may be configured to compute the resistance based on the measured voltage and/or to determine, e.g., a strain, an extension, or a property derived from the strain or extension, based on the computed or measured electrical resistance. If the sensor has a high linearity, the strain ε may be determined by computing:

$$ϵ = \frac{1}{G} \frac{Δ R}{{R}_{0}} \qquad ( 4 )$$

where G is de gain factor, R₀ is the initial resistance (usually corresponding to ε=0), and ΔR=R−R₀ is the change in resistance.

Alternatively, and particularly for sensors with a lower linearity, the strain may be determined based, e.g., on look-up tables, or on a fitted function.

FIG. 7A-7C schematically depict sensor elements according to embodiments of the invention. FIG. 7A depicts a sensor element comprising a single knitted sensor line segment 702. The sensor line may comprise 1 to 20 adjacent conductive courses as described above. The sensor line may be embedded in an elastic matrix 704 comprising a plurality of courses of a non-conductive elastic yarn with a high recovery on either side of the sensor line. The width of the elastic matrix on either side of the sensor line may be at least 2 mm, e.g., at least 5 mm. The elastic matrix may comprise at least four courses, e.g., at least eight, at least twelve, or even at least sixteen courses on either side of the sensor line. The elastic matrix can optionally be knitted using a plated knitting technique using an elastic yarn, e.g., the same elastic yarn, forming both the core and the surfaces of the fabric (i.e., being positioned both on the inside and on the outside). The sensor line comprises two electrical connection points 706_{1,2}, one on either end of the sensor line. The electrical connection points may be used to connect the sensor line to, e.g., a measurement unit of a sensor. A single-line sensor element is easy to construct.

FIG. 7B depicts a sensor element comprising two knitted sensor line segments 712_{1,2}. Each line segment may comprise 1 to 20 adjacent conductive courses. Typically, the line segments comprise an equal number of adjacent courses. The sensor line segments may be separated by a plurality of courses of a non-conducting yarn, the plurality generally comprising at least four, at least eight, at least twelve, or at least sixteen courses. The sensor line segments are electrically connected by a conductive interconnection line 718. The interconnection line may be knitted into or stitched onto the elastic matrix, but in principle, any type of electric connection can be used. The interconnection line may have a high conductivity (low resistivity), and no or only a negligible reaction to strain. A sensor element with two sensor line segments can cover a larger surface than a single-line sensor element. Additionally, the electrical connection points may be placed close together, allowing easy connection to a measurement unit.

FIG. 7C depicts a sensor element comprising multiple knitted sensor line segments 712₁₋₄ connected using interconnection lines 718₁₋₃. The depicted embodiment comprises four line segments, but in principle, any number of line segments may be used. If an even number of line segments is used, the electrical connection points 706_{1,2} are located on the same side of the sensor element, which can be advantageous. With a sensor element with multiple line segments, an arbitrarily large surface may be sensed, depending on the number of line segments.

FIG. 8 schematically depicts a garment comprising an embedded knitted strain sensor element according to an embodiment of the invention. The garment 800 may be an elastic, knitted, close-fitting garment, e.g. a shirt (depicted), pants, bra, stockings, gloves, et cetera. The garment can also be a separately applied band. The garment may comprise a thoracal sensor 802 with single-line sensor element. This sensor may be used to monitor, e.g., breathing. The garment further comprises two elbow sensors 804, 806 with two-line sensor elements, positioned on the outside of the elbows. As the sensor elements are flexible, they can be bent without substantial detrimental effect. By determining the extension of the sensor, the angle of the elbow can be determined. Monitoring of elbow positions typically requires a working range of about 30-40%-point. The garment further comprises an abdominal sensor 808 with a multi-line sensor element covering a large area of the abdomen. The abdominal sensor may be used, for instance, in conjunction with the thoracal sensor and a feedback element to train or assist a user with abdominal breathing. One or more of the sensors can share sensor hardware such as a power source (e.g., a battery) and a processing unit. Alternatively, each sensor may comprise its own hardware.

Evidently, only a few of the many possible applications are depicted here. Depending on the signal to be monitored, a different garment may be selected. For example, a respiration monitoring sensor can be provided either as a separate band, integrated in under garments, e.g. in a bra, or in outer garments, e.g. a sports shirt. Stockings, in particular compression stockings, may be used to monitor limb compression. A knee or elbow brace may be equipped with an integrated knitted sensor for monitoring stretching angles during revalidation exercises. Similarly, a ‘smart’ fitness shirt with knitted strain sensors can be used for exercise monitoring and feedback. A feedback unit may be configured to provide feedback based on sensor signals and, typically, one or more predefined criteria. This way, the user can, e.g., track progress or check whether the exercises are being performed correctly.

In a sports context, knitted sensors may be used for monitoring limb or upper body movements, possibly in combination with other sensors such as accelerometer. Similar sensors or combinations of sensors may be used to determine motion and/or posture to be represented in a virtual reality context. Knitted strain sensors for posture monitoring may also be used for unobtrusive monitoring of posture during everyday life situations for health applications (e.g., keeping you back straight) or for seating comfort monitoring during long travelling (e.g., in an airplane or a car). A connected feedback system may provide feedback to, e.g., warn a user of bad posture.

The knitted strain sensors described in this application are generally comfortable and breathable, washable, durable, and reliable (also after repeated washing), making them suitable for these and many more applications.

The depicted garment is intended for human use. Other types of garments or garment-like objects such as sleeves may be used for other applications. For example, animal motions may be monitored for, e.g., health care, physiotherapy, sports, or scientific research. Robots can be equipped with integrated or applied knitted sensors to monitor the position of bendable and/or extendable parts.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

1. A knitted strain sensor element comprising an electrically conducting yarn and an elastic yarn, the elastic yarn having a Young's modulus that is substantially lower than the electrically conducting yarn's Young's modulus;
wherein the knitted strain sensor element is knitted using a knit stitch pattern comprising knitted stitches and purled stitches on each course; and
wherein the electrically conducting yarn and the elastic yarn are knitted together using a plated knitting technique forming a knitted fabric, the electrically conducting yarn forming a core of the knitted fabric and the elastic yarn forming surfaces of the knitted fabric.

2. The knitted strain sensor element as claimed in claim 1, wherein the knitted strain sensor element comprises one or more adjacent courses of the electrically conducting yarn.

3. The knitted strain sensor element as claimed in claim 2, further comprising one or more adjacent courses of the elastic yarn on either side of the one or more courses of the electrically conducting yarn.

4. The knitted strain sensor element as claimed in claim 1, wherein the electrically conducting yarn has an electrical resistance in the range of 5-10000 Ω/m inclusive.

5. The knitted strain sensor element as claimed in claim 1, wherein the electrically conducting yarn comprises a non-conductive core with an electrically conductive coating.

6. The knitted strain sensor element as claimed in claim 1, wherein the elastic yarn has an elastic recovery of more than 95%.

7. The knitted strain sensor element as claimed in claim 1, wherein the elastic yarn comprises a synthetic polyamide and/or a synthetic polyether-polyurea copolymer.

8. A strain sensor comprising a knitted strain sensor element as claimed in claim 1, a measurement unit for measuring the electrical resistance of the knitted strain sensor element, and a processing unit coupled to the measurement unit, the processing unit being configured to determine a strain or a property derived from the strain, based on the measured electrical resistance.

9. A textile comprising a knitted strain sensor element as claimed in claim 1, the knitted strain sensor element being embedded in the textile.

10. A garment comprising a knitted strain sensor element as claimed in claim 1, wherein the knitted strain sensor element is adapted for sensing a physiological signal.

11. A garment comprising a knitted strain sensor element as claimed in claim 1, wherein the knitted strain sensor element is adapted for determining a position of a body part.

12. The knitted strain sensor element as claimed in claim 1, wherein the knit stitch pattern is a rib stitch pattern.

13. The knitted strain sensor element as claimed in claim 12, wherein the rib stitch pattern is a 1×1 rib stitch pattern.

14. The knitted strain sensor element as claimed in claim 2, wherein the knitted strain sensor element comprises at most twenty adjacent courses of the electrically conducting yarn.

15. The knitted strain sensor element as claimed in claim 3, wherein the knitted strain sensor element comprises five or more adjacent courses of the elastic yarn on either side of the one or more courses of the electrically conducting yarn.

16. The knitted strain sensor element as claimed in claim 4, wherein the electrically conducting yarn has an electrical resistance in the range of 10-1000 Ω/m inclusive.

17. The knitted strain sensor element as claimed in claim 5, wherein the coating comprises silver and the non-conductive core comprises a synthetic polyamide or a polyester.

18. The knitted strain sensor element as claimed in claim 6, wherein the elastic yarn has an elastic recovery of substantially 100%.

19. The knitted strain sensor element as claimed in claim 7, wherein the elastic yarn comprises between 10-25% inclusive fibers of the synthetic polyether-polyurea copolymer.

20. The garment as claimed in claim 10, wherein the physiological signal is one of: a respiration signal, a limb compression, a skin deformation, a stretching, or a body motion.
